(19) United States
(12) Patent Application Publication
(10) Pub. No.: US 2005/0123560 A1
(43) Pub. Date: Jun. 9, 2005

(54) High purity and water dispersible extract and formulations of larrea tridentata leaf resin, and methods of making and using the same

(76) Inventor: Robert A. Sinnott, Chandler, AZ (US)

Correspondence Address: INGRASSIA FISHER & LORENZ, P.C., 7150 E. CAMELBACK, STE. 325, SCOTTSDALE, AZ, 85251, US

(21) Appl. No.: 10/932,616

(22) Filed: Sep. 1, 2004

Related U.S. Application Data
(60) Provisional application No. 60/527,322, filed on Dec. 4, 2003.

(51) Int. Cl. A61K 35/78

(52) U.S. Cl. 424/195.18; 424/769

(57) ABSTRACT

A method for preparing a nontoxic composition that includes an extract of Larrea tridentata plant material and is substantially free of nordihydroguaiaretic acid (NDGA) quinone includes the steps of mixing the Larrea tridentata plant material with an aqueous solvent to produce an extract, and reducing any NDGA quinone in the extract to NDGA. A method for treating an infection from a virus includes the step of administering to a human in need thereof a pharmaceutical composition comprising an extract of Larrea tridentata in an amount that is effective to suppress NF-kB activation. An antimicrobial solution includes an aqueous solvent, and an extract of Larrea tridentata plant material. A formulation for medical and health products includes an extract of Larrea tridentata plant material, and at least one detoxification enhancer that functions as a chemoprotective agent.

### CROSS-REFERENCES TO RELATED APPLICATIONS

This application claims the benefit of U.S. Provisional Application No. 60/527,322, filed Dec. 4, 2003.

### TECHNICAL FIELD

The present invention relates generally to extracts and formulations of Larrea tridentata plant material, along with methods for making and using the same.

### BACKGROUND

Larrea tridentata, also known as Larrea divaricata, Larrea, chaparral, or creosote bush, hereinafter referred to as “Larrea,” is a shrubby plant that dominates some desert areas of the southwestern United States, Northern Mexico, and Argentina. Tea made from the Larrea leaves has long been used in folk medicine to treat digestive disorders, rheumatism, venereal disease, sores, bronchitis, chicken pox, and the common cold.

Resin on the Larrea leaf surface constitutes approximately 10-15% of the dry leaf weight and is composed of approximately 50% nordihydroguaiaretic acid (“NDGA”) and related lignans, and approximately 50% flavonoids. By far, NDGA is the dominant lignan in the external leaf resin, although NDGA dimethyl ether (dihydroguaiaretic acid) and NDGA monomethyl ether (partially demethylated dihydroguaiaretic acid) occur in significant quantities as well. The complex flavonoid fraction of the external leaf resin includes predominantly methyl ethers of flavones, flavanols, and dihydroflavanols. Specifically, the major flavonoid components are quercetin (as the flavonoid aglycone and mono-, di-, tri-, and tetra-methyl ethers), gossypetin (as di- and tri-methyl ethers), kaempferol (as the flavonoid aglycone and mono- and di-methyl ethers), gossypetin (as di- and tri-methyl ethers), apigenin (as the flavonoid aglycone and monomethyl ether), and herbacetin (as the dimethyl ether).

NDGA is a powerful antioxidant, and has been extracted from Larrea by an alkaline extraction method such as that described in U.S. Pat. No. 2,382,475. NDGA can also be produced synthetically using methods such as those described in U.S. Pat. No. 2,644,822. Because of its antioxidant properties, NDGA was used as an additive in edible fats, butter, oils and oleaginous materials, as described in U.S. Pat. No. 2,373,192, until the GRAS (Generally Recognized As Safe) status of NDGA was revoked after animal studies revealed evidence of kidney toxicity resulting from the ingestion of NDGA.

The apparent toxicity related to NDGA is not from the compound itself, but rather due to products resulting from reactions between NDGA and oxidizing chemicals that are exposed to the NDGA within the Larrea plant material, and also during processing and storage. Studies have confirmed that oxidation products of NDGA are found in Larrea, and are suspected to be causative agents of the toxic effects associated with consumption of Larrea products. A highly reactive and toxic oxidation product of NDGA is nordihydroguaiaretic acid ortho di-a-b-unsaturated quinone (NDGA quinone) which has been found to occur in Larrea and Larrea extracts and is thought to serve as a toxin to protect the plant from being eaten by herbivores.

Despite the alleged dangers associated with NDGA quinone, NDGA is known to possess wide ranging beneficial biological activity including anti-tumor activity, enzyme inhibition activity, antimicrobial activity, and antiviral activity. Consequently, there has been a need for a method of producing a Larrea extract that contains a high concentration of NDGA and other antiviral lignans, flavonoids, and a wide variety of other associated organic compounds from the Larrea leaf resin. Formulations, production methods, and uses for such an extract are disclosed in U.S. Pat. Nos. 5,837,252; 5,945,106; 6,004,559; and 6,039,955. Although the Larrea extract and the formulations described in these patents are essentially free of NDGA quinone and are useful for many applications, they are somewhat expensive to make. Also, the extract tends to have low water solubility, rendering the extract difficult to incorporate into some types of cosmetics, pharmaceuticals, cleaning products, foods, and other products.

In light of the foregoing background, there is a need for Larrea leaf resin extracts that have high purity and water dispersibility. There is also a need for an economical method to commercially produce such an extract. Additionally, there is a need for a medicinal Larrea extract that is processed in a manner that reduces the concentration of the toxic compounds such as NDGA quinone. There is also a need to inhibit the natural production of such toxic oxidation products in the Larrea extract during processing and storage of the extract and formulated products, and to facilitate processing of the concentrated extract in many different types of formulations. Furthermore, other desirable features and characteristics of the present invention will become apparent from the subsequent detailed description and the appended claims, taken in conjunction with the foregoing technical field and background.

### BRIEF SUMMARY

A method is provided for preparing a nontoxic composition that includes an extract of Larrea tridentata plant material and is substantially free of nordihydroguaiaretic acid (NDGA) quinone. The method comprises the steps of mixing the Larrea tridentata plant material with an aqueous solvent to produce an extract, and reducing any NDGA quinone in the extract to NDGA.

According to one embodiment, the method further comprises the steps of separating the extract from the Larrea tridentata plant material, rinsing the separated Larrea tridentata plant material with an aqueous solution comprising at least one component selected from the group consisting of a reducing agent, an alkaline pH adjusting agent, and a detergent, and collecting the aqueous solution with the extract after rinsing the Larrea tridentata plant material.

A method is also provided for treating an infection from a virus that has replication affected by transcription nuclear factor-kappa beta (NF-kB) activation. The method comprises the step of administering to a human in need thereof a pharmaceutical composition comprising an extract of Larrea tridentata in an amount that is effective to suppress NF-kB activation.

An antimicrobial solution is also provided. The solution comprises an aqueous solvent, and an extract of Larrea tridentata plant material.

A formulation is also provided for medical and health products. The formulation comprises an extract of Larrea tridentata plant material, and at least one detoxification enhancer that functions as a chemoprotective agent.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

The present invention satisfies the need for a high purity and nontoxic Larrea leaf resin extract that has good water dispersibility, and also satisfies the need for an economical method to commercially produce the extract and formulations thereof. In addition, the present invention includes many new and advantageous uses for the Larrea leaf resin extract.

To start, a method of preparing the extract will be described, beginning with a process for preparing air-dried Larrea plant material. Foliage that mainly comprises leaves and some stems from Larrea shrubs is manually or mechanically harvested, and then air-dried on well ventilated racks, preferably in the shade and at a temperature that does not exceed 50° C. In order to provide foliage that mainly comprises leaves, the dried plant material can be processed by gentle compression to separate the foliage from large stems. Processing should be carefully carried out in order to keep the leaves from appreciably breaking up. Further processing can be carried out, including screening the dried foliage to separate the leaves from the plant stems. In an exemplary process, separation is performed by placing the dried foliage on ½ and/or 1/4 inch mesh screen. Large stems are then discarded, leaving the dried and screened plant material which is almost entirely leaves.

After drying and screening is completed, leaf resins are extracted. A non-toxic, polar extraction solvent with added antioxidants and/or reducing agents is employed in an exemplary embodiment. An aqueous solvent is preferred, although additional water miscible solvents such as FCC or USP grade propylene glycol, glycerin, and/or ethanol can be included. Exemplary antioxidants and/or reducing agents include FCC or NF grade butylated hydroxytoluene (BHT) at approximately 1.0 g/L and/or FCC or USP grade ascorbic acid powder at approximately 20.0 g/L, with ascorbic acid being preferred.

An important principle of one exemplary embodiment of the current invention is that ascorbic acid is combined with NDGA and reduces any NDGA quinone present into NDGA. Ascorbic acid also effectively prevents NDGA from oxidizing and producing more NDGA quinone. Other compounds and agents may be theoretically used to reduce NDGA, and to prevent oxidation of NDGA into an oxidation product. Such agents include ascorbic acid esters (i.e. ascorbyl palmitate), ascorbic acid salts (i.e. sodium ascorbate), butylated hydroxyanisole (BHA), butylated hydroxytoluene (BHT), hydrogen sulfide, hypophosphorous acid (phosphinic acid), monothioglycerol (3 mercapto-1,2-propanediol), potassium bisulfite (potassium metabisulfite, potassium pyrosulfite), propyl gallate, sodium bisulfite (sodium metabisulfite, sodium pyrosulfite), sodium hydrosulfite (sodium dithionite), sodium thiosulfate (sodium hyposulfite), sulfur dioxide, sulfurous acid, a tocopherol, or vitamin E (DL-alpha-tocopherol). Such reducing agents are not necessarily considered to be equivalents to ascorbic acid or to each other, some having advantages not possessed by all the others.

Ascorbic acid has been determined to be an unexpectedly powerful reducing agent for converting NDGA quinone to NDGA. In laboratory tests, ascorbic acid, sodium hydrosulfite, and sodium bisulfite were added at 100 mg/ml to separate methanolic solutions of oxidized NDGA. Ascorbic acid proved to be the fastest reducing agent (approximately 1 minute). Subsequent chromatography of the mixtures showed that when sodium hydrosulfite or sodium bisulfite is used as the reducing agent, NDGA quinone is still detectable. However, when ascorbic acid is used as a reducing agent, all traces of NDGA quinone are completely eliminated. Accordingly, ascorbic acid proved to be a better reducing agent than either sodium bisulfite or sodium hydrosulfite. This is an unexpected advantage since both sodium bisulfite and sodium hydrosulfite are strong reducing agents with reducing power considered as effective or more effective than the reducing power of ascorbic acid. In fact, sodium hydrosulfite has almost ten times the reduction potential of ascorbic acid. Thus, a nontoxic extract of Larrea having a high concentration of NDGA and very little or no NDGA quinone can be prepared according to the principles of the present invention by adding ascorbic acid during the extraction step. Further, saturating the extract with ascorbic acid after the extraction step ensures that NDGA quinone will not form as an oxidation product in the stored extract or formulations thereof.

Returning now to the extraction process, a thorough and substantially complete extraction is accomplished by mixing the extraction solvent and the dried, screened plant material in a closed container or drum. In an exemplary embodiment, the extraction solvent is heated before being added to the plant material to facilitate faster and more complete extraction of leaf resins. For example, if the extraction solvent includes water, the extraction solvent may be heated to approximately 50 to 100° C. before adding the extraction solvent to the plant material. Approximately 1.0 liter of extraction solvent is used to extract each kilogram of air-dried plant material. Mixing can be carried out by rolling the container or drum containing the plant material and the solvent. In an exemplary embodiment, the plant material and solvent are rolled at approximately 20 to 30 rpm for approximately 2 to 6 hours.

After the extraction process is complete, the solvent is removed from the plant material by, for example, decanting the solvent. Approximately half of the original volume of the extraction solvent can be easily recovered by simple draining and decanting from foliage for several hours. If it is necessary to filter the decanted solvent, a suitable straining device such as stainless steel wire mesh can be used to remove small plant parts and other coarse debris. Small suspended particles can be removed by passing the solvent through a suitable fine filter, such as polypropylene felt particulate filter (e.g. 5 micron pore size). The resulting foliage extract is typically transparent amber to dark amber in color, depending on the concentration of leaf resin chemicals in the extract. The extract has a viscosity similar to the extraction solvent that was employed for the extraction, and is readily water dispersible.

In order to further ensure that the foliage extract does not oxidize during further processing or storage, the extract can be passed through a bed of ascorbic acid powder. In one example, 5 grams of FCC grade ascorbic acid powder for each liter of extract, although the exact ascorbic acid concentration is less important than the principle of saturating the extract with the ascorbic acid. In an exemplary embodiment, the extract is passed through the ascorbic acid bed in a manner that facilitates contact between the extract and the ascorbic acid powder, and saturates the extract with ascorbic acid. Saturating the extract with the ascorbic acid results in conditions that favor the chemical reduction of any oxidative metabolites of NDGA that may be present in Larrea plant tissues and the resulting extracts, and further prevents such oxidative metabolites from forming.

Next, each production lot of foliage extract is assayed and standardized to bring the resin in the solution to a known concentration. In one embodiment, assaying is performed by quantitatively diluting one or more random production lot samples in a suitable solvent, such as methanol, and using a spectrophotometer to measure absorbance of the sample at 370 nm. Absorbance measurements are compared against absorbance for a Larrea leaf resin solution at a known resin concentration to easily determine the resin concentration in each production lot being assayed.

Depending on the resin concentration in each sample, the corresponding production lot may be diluted with additional amounts of water, other solvents, and/or other suitable ingredients to bring the final concentration to a known concentration endpoint. In just one example, the solution is standardized to about 20% resin in solvent on a weight/weight basis.

Once the leaf resin concentration is standardized, the aqueous extract with a known resin concentration can be packaged and stored. Tightly sealed containers made from polypropylene, polyethylene, glass or other suitable materials are suitable for storing and shipping the extract. Using previous extraction methods, storage and shipping were performed after the time consuming and costly process of evaporating solvent from the resin solution. The ability to store and ship the standardized solution as an aqueous solution is an important processing advantage that provides significant economic, safety and environmental advantages.

Next, an extraction method that includes a centrifuging step will be described, although the solvents and other chemicals used in this extraction method are the same as those previously discussed, unless otherwise noted. According to the previously described embodiment, approximately half of the original extraction solvent volume can be recovered by simple decanting and draining from foliage to produce a high quality, water dispersible extract. According to this second embodiment, solvent recovery from the foliage is greatly enhanced by centrifuging the solvent/foliage mixture in a centrifugal dryer unit.

After removing the extraction solvent by either decanting and draining or by centrifuging the solvent/foliage mixture, some of the extract solution containing aqueous extraction solvent and solubilized Larrea leaf resin remains absorbed on the foliage. Substantially all of the remaining extraction solvent, including the solubilized Larrea leaf resin bound to the extracted foliage, is recovered by rinsing the foliage with an aqueous solution containing a reducing agent such as ascorbic acid, and/or an alkaline pH adjusting agent such as sodium hydroxide, and/or a suitable detergent such as polysorbate 80. The types and concentrations of the reducing agent, pH adjusting agent, and detergent can be modified according to need, and in some cases one or more of these components may not be necessary at all. The aqueous solution may be optionally heated to facilitate complete removal of the extraction solvent and solubilized leaf resin components from the foliage surfaces. Approximately one to two liters of aqueous solution is typically used for each kilogram of dried, screened Larrea foliage initially extracted in the above-described procedure. The aqueous solution containing the solubilized Larrea leaf resin may be filtered if necessary before subsequent processing steps.

If desired, the solubilized Larrea leaf resins may be recovered from the aqueous solution by precipitation and/or filtration. Precipitation may be facilitated by one or more procedures including cooling the aqueous solution, adjusting the pH of the aqueous solution to an acidic state, and adding a suitable water soluble salt such as sodium chloride to the aqueous solution. The precipitated or filtered product may be washed several times with water or an aqueous solution containing a suitable antioxidant compound such as ascorbic acid to remove undesirable, water-soluble compounds. The high purity Larrea leaf resin product may then be collected, packaged and stored in tightly sealed containers made from polypropylene, polyethylene, glass, or other suitable materials. Additionally, for convenience of producing final products containing this Larrea leaf resin extract and/or to facilitate enhanced storage stability of said extract, the extract may be diluted in a suitable solvent, such as an aqueous solvent, and/or compounded with additional antioxidant ingredients. As mentioned previously, additional amounts of ascorbic acid are added to the extract or products formulated therefrom in an exemplary embodiment of the invention during storage, as well as during previous processing steps, to inhibit the natural oxidation of component NDGA into NDGA quinone.

Having provided exemplary methods of making a nontoxic extract of Larrea leaf resin, formulations of the same will now be discussed, focusing on the therapeutic properties of the extract and its formulations. Some of the therapeutic components in the Larrea leaf resin extract include lignans and flavonoids, which are small, hydrophobic phenylic compounds that readily diffuse through cell membranes. Many of the flavonoid and lignan components occurring in the Larrea leaf resin have been shown to possess powerful antiviral, antimicrobial and anti-inflammatory activities in many test systems. Certain flavonoid compounds, especially members of the flavone and flavonol chemical classes, can inhibit viral activation of HIV and other viruses at fairly low concentrations. Flavones and flavonols occur at high concentration in the Larrea leaf resin. Further, the lignan components occurring in the Larrea leaf resin have also been shown to have powerful anti-viral and anti-inflammatory properties. Like many physiologically active compounds derived from plant sources, the flavonoid and lignan components of the Larrea leaf resin may contribute significantly, and possibly synergistically, to the anti-viral, anti-inflammatory, and anti-microbial effects exhibited by Larrea.

Some of the antioxidant phenylic compounds that are prevalent in Larrea leaf resin extract, particularly the flavonoid compounds, are powerful inhibitors of the redox-sensitive transcription factors such as nuclear factor-kappa beta (NF-kB). See Hill et al., Antioxidants Attenuate Nuclear Factor-kappa B Activation and Tumor Necrosis Factor-alpha Production in Alcoholic Hepatitis Patient Monocytes and Rat Kupffer Cells, In Vitro, Clin. Biochem. Vol. 32, No. 7, 563-570 (1999); Rangan et al., Inhibition of NFkB activation with antioxidants is correlated with reduced cytokine transcription in PTC, Am. J. Of Phys. 277, (Renal Phys. 46), F779-F789 (1999); and Liang et al., Suppression of Inducible Cyclooxygenase and Inducible Nitric Oxide Synthase by Apigenin and Related Flavonoids in Mouse Macrophages, Carcinogenesis Vol. 20, No. 10, 1945-1952 (1999). Consequently, the present invention includes methods for treating a class of viral diseases having their transcription initiation and/or progression affected by redox-sensitive transcription factors such as NF-KB, including respiratory syncytial virus (RSV) of the family Paramyxoviridae (Carpenter et al., Respiratory Syncytial Virus and TNFalpha Induction of Chemokine Gene Expression Involves Differential Activation of Rel A and NF-kappaB1, BMC Infectious Diseases Vol. 2, No. 5 (2002)), Influenza virus of the family Orthomyxoviridae (Flory et al., Influenza Virus-induced NF-kB-dependent Gene Expression Is Mediated by Overexpression of Viral Proteins and Involves Oxidative Radicals and Activation of IkB Kinase, J. Biol. Chem. Vol. 275, No. 12, 8307-8314 (2000)), Adenovirus of the family Adenoviridae (Deryckere et al., Tumor Necrosis Factor (alpha) Induces the Adenovirus Early 3 Promoter by Activation of NF-kB, J. Biol. Chem. Vol. 271, No. 47, 30249-30255 (1996)), Rhinovirus of the family Picorniviridae (Zhu et al., Rhinovirus stimulation of Interleukin-8 In Vivo and In Vitro: Role of NF-kB, Am. J. Phys. 273 (Lung Cell. and Molec. Phys. 17), L814-L824 (1997)), Hepatitis B virus of the family Hepadnaviridae (Ohata et al., Interferon Alpha Inhibits the Nuclear Factor Kappa B Activation Triggered by X Gene Product of Hepatitis B Virus in Human Hepatoma Cells, FEBS Letters Vol. 553, 304-308 (2003)), and Hepatitis C virus of the family Flaviviridae (de. Lucas et al., Hepatitis C Virus. Core Protein Transactivates the Inducible Nitric Oxide Synthase Promoter via NF-kB Activation, Antiviral Research Vol. 60, 117-124 (2003); Ray et al., Distinct Functional Role of Hepatitis C Virus Core Protein on NF-kB Regulation is Linked to Genomic Variation, Virus Research Vol. 87, 21-29 (2002); Shimotohno, Hepatitis C and Its Pathogenesis, Seminars in Cancer Biol. Vol. 10, 233-240 (2000)). Additionally, as applies to hepatitis and liver disease, inhibition of NF-KB has been suggested to be an attractive potential therapy for liver disease (Heyninck et al., Nuclear Factor-kappa B Plays a Central Role in Tumor Necrosis Factor-mediated Liver Disease, Biochem. Pharm. Vol. 66, 1409-1415 (2003)).

Also, the Larrea resin extract according to the present invention has significant antimicrobial activity that may be exploited commercially in a wide range of products. For instance, it has been found by the current inventor that at approximately 1% weight/weight concentration, purified Larrea leaf resin extract inhibits the growth of all gram negative and gram positive bacteria and yeasts included in the United States Pharmacopeia (USP) preservative efficacy test battery, including Staphylococcus aureus, E. coli, Pseudomonas aeruginosa, Candida albicans, and Aspergillis niger. Because the Larrea resin extract of the present invention has these antimicrobial properties, the extract is suitable for inclusion in a wide variety of consumer and industrial products including, soaps, detergents, disinfectants, sanitizers, cosmetics, external hygiene products, oral hygiene products, dental products, shampoos, deodorants, dietary supplements, pharmaceuticals including OTC and prescription drugs, medical devices, nutriceuticals, cosmeceuticals, sanitary products, pest control products, and other home and garden products.

To produce cleaning and disinfecting products, Larrea leaf resin extract prepared according to the method of the present invention may be formulated with suitable solvents and/or surfactants and/or other antimicrobial ingredients to enhance cleaning and antimicrobial efficacy. Some suitable ingredients for this purpose include isopropanol, ethanol, propanol, butanol, propylene glycol, ethylene glycol, polyethylene glycol, polypropylene glycol, butylene glycol, 2-alkyl-N-carboxymethyl-N-hydroxyethyl imidazolinium betaine, alkyl alkanol amide, alkyl benzyl dimethyl ammonium chloride, ammonium lauryl sulfate, cetyl trimethyl ammonium chloride, coconut amine acetate, distearyl dimethyl ammonium chloride, ethylene glycol distearate, fatty acid alkanol amide, glycerol monooleate, lauryl betaine, lauryl dimethyl amine oxide, lauryl imidazolinium betaine, lauryl trimethyl ammonium chloride, mono- and diglyceride, polyethylene glycol distearate, polyethylene glycol monooleate, polyethylene glycol monolaurate, polyethylene glycol monostearate, polyoxyalkylene alkylether, polyoxyethylene alkyl amine, polyoxyethylene alkyl ether, polyoxyethylene cetyl ether, polyoxyethylene derivatives, polyoxyethylene hydrogenated castol oil, polyoxyethylene lauryl ether, polyoxyethylene oleyl ether, polyoxyethylene sorbitan mono coconut fatty acid ester, polyoxyethylene sorbitan monolaurate, polyoxyethylene sorbitan monooleate, polyoxyethylene sorbitan monopalmitate, polyoxyethylene sorbitan monostearate, polyoxyethylene sorbitan triisostearate, polyoxyethylene sorbitan trioleate, polyoxyethylene sorbitan tristearate, polyoxyethylene sorbitol tetraoleate, polyoxyethylene stearyl ether, potassium hydroxy stearate, potassium oleate, potassium polyoxyethylene alkyl ether phosphate, potassium salt of castor oil, potassium soap of partially hydrogenated tallow fatty acid, sodium alkane sulfonate, sodium alkyl diphenyl disulfonate, sodium alkyl naphthalene sulfonate, sodium arylsulfonate formaldehyde condensate, sodium beta Naphthalene sulfonate formaldehyde condensate, sodium dialkyl sulfosuccinate, sodium dodecylbenzene sulfonate, sodium fatty alcohol sulfate, sodium lauryl sulfate, sodium polyoxyethylene alkylether sulfate, sodium polyoxyethylene lauryl ether sulfate, sodium soap of blended fatty acid, sodium soap of partially hydrogenated tallow fatty acid, sodium stearate, sorbitan distearate, sorbitan monolaurate, sorbitan monooleate, sorbitan monopalmitate, sorbitan monostearate, sorbitan sesquioleate, sorbitan trioleate, sorbitan tristearate, stearyl amine acetate, stearyl trimethyl ammonium chloride, triethanolamine lauryl sulfate, and triethanolamine polyoxyethylene alkylether sulfate.

To produce medical and health products, including dietary supplements, OTC drugs, prescription drugs, medical devices, nutriceuticals, and cosmeceuticals including the Larrea leaf resin extract prepared according to the method of the present invention, the extract can be formulated with additional ingredients to impart desired additional biochemical properties for specific purposes. These additional ingredients can include, for example, detoxification enhancers to function as chemoprotective agents. Compounds suitable for this purpose include diindolymethane, indole-3-carbinol, methionine, methionine derivatives, cysteine, cysteine derivatives, N-acetyl cysteine, cystine, methylsulfonylmethane (MSM), flavonoids, milk thistle extract, silymarin, sylbinin, benzothiazole, garlic extract, propolis, tannic acid, ellagic acid, coumarin, turmeric extract, curcumin, allyl isothiocyanate, benzyl isothiocyanate, phenethyl isothiocyanate, benzyl thiocyanate, brassica extracts, cruciferous vegetable extracts, broccoli extract, brussel sprout extract, sulforaphane, sulforaphane nitrile, glucosinolates, green tea extract, epigallocatechin gallate (EGCG), tannic acid, ginkgo extract, resveratrol, grape extract, selenium, selenomethionine, glutathione, lipoic acid, coenzyme Q, oleuropein, hops extract, humulone and/or synthetic phenylic antioxidants such as BHA, BHT, TBHQ and hydroquinone. See Jeong et al., Modulatory Properties of Various Natural Chemopreventative Agents on the Activation of NF-kappaB Signaling Pathway, Pharm. Research Vol. 21, No. 4, 661-670 (2004); Schumann et al., Silibinin Protects Mice from T Cell-dependent Liver Injury, J. Hepatology Vol. 39, 333-340 (2003); Krajka-Kuzniak et al., The Effects of Tannic Acid on Cytochrome P450 and Phase II Enzymes in Mouse Liver and Kidney, Toxicology Letters Vol. 143, 209-216 (2003); Spencer et al., Intracellular Metabolism and Bioactivity of Quercetin and Its In Vivo Metabolites, Biochem. J. Vol. 372, 173-181 (2003); van der Logt et al., Induction of Rat Hepatic and Intestinal UDP-glucuronosyltransferases by Naturally Occurring Dietary Anticarcinogens, Carcinogenesis Vol. 24, No. 10, 1651-1656 (2003); Won Seo et al., Effects of Benzothiazole on the Xenobiotic Metabolizing Enzymes and Metabolism of Acetaminophen, J. App. Toxicology Vol. 20, 427-430 (2000); Sasaki et al., Effects of Extract of Ginkgo Biloba Leaves and Its Constituents on Carcinogen-metabolizing Enzyme Activities and Glutathione Levels in Mouse Liver, Life Sciences Vol. 70, 1657-1667 (2002); Won Nho et al., The Synergistic Upregulation of Phase II Detoxification Enzymes by Glucosinolate Breakdown Products in Cruciferous Vegetables, Toxicology and App. Pharm. Vol. 174, 146-152 (2001).

Additionally, certain formulations may include one or more stimulant ingredients such as coffee extract, gotu kola, cola extract, caffeine, cocoa extract, theobromine, tea extract, theophylline, synephrine, ginseng, taurine, ephedrine, and pseudoephedrine.

Another use for the Larrea leaf resin extract prepared using the method of the present invention includes the production of building materials, other materials having a cellulose base such as wood, paper, and building material treatments with antimicrobial and preservative properties to prevent infestation and damage by animals, insects, fungi, bacteria, etc. According to this embodiment of the invention, naturally derived Larrea leaf resin extract is used to replace common toxic building material treatments such as inorganic arsenical pressure-treated wood (CCA), ammoniacal copper arsenate (ACA), ammoniacal copper zinc arsenate (ACZA), acid copper chromate (ACC), chromated zinc chloride (CZC), pentachlorophenyl pressure-treated wood and creosote pressure-treated wood. Formulation for the building materials and building material treatments can also include ingredients such as urethane, shellac, latex, epoxy, enamel, varnish, coal tar pitch and coal tar pitch emulsion, pigments, polymers, and various solvents.

A similar product utilizing the Larrea leaf resin extract prepared using the method of the present invention is a formulation for preventing infestation of animals, insects, fungi, and bacteria. The formulation includes an extract of Larrea plant material, and at least one aqueous solvent. Additional ingredients can be added, including isopropanol, ethanol, propanol, butanol, propylene glycol, ethylene glycol, polyethylene glycol, polypropylene glycol, butylene glycol, wood rosin, 2-alkyl-N-carboxymethyl-N-hydroxyethyl imidazolinium betaine, alkyl alkanol amide, alkyl benzyl dimethyl ammonium chloride, ammonium lauryl sulfate, cetyl trimethyl ammonium chloride, coconut amine acetate, distearyl dimethyl ammonium chloride, ethylene glycol distearate, fatty acid alkanol amide, glycerol monooleate, lauryl betaine, lauryl dimethyl amine oxide, lauryl imidazolinium betaine, lauryl trimethyl ammonium chloride, mono- and diglyceride, polyethylene glycol distearate, polyethylene glycol monooleate, polyethylene glycol monolaurate, polyethylene glycol monostearate, polyoxyalkylene alkylether, polyoxyethylene alkyl amine, polyoxyethylene alkyl ether, polyoxyethylene cetyl ether, polyoxyethylene derivatives, polyoxyethylene hydrogenated castol oil, polyoxyethylene lauryl ether, polyoxyethylene oleyl ether, polyoxyethylene sorbitan mono coconut fatty acid ester, polyoxyethylene sorbitan monolaurate, polyoxyethylene sorbitan monooleate, polyoxyethylene sorbitan monopalmitate, polyoxyethylene sorbitan monostearate, polyoxyethylene sorbitan triisostearate, polyoxyethylene sorbitan trioleate, polyoxyethylene sorbitan tristearate, polyoxyethylene sorbitol tetraoleate, polyoxyethylene stearyl ether, potassium hydroxy stearate, potassium oleate, potassium polyoxyethylene alkyl ether phosphate, potassium salt of castor oil, potassium soap of partially hydrogenated tallow fatty acid, sodium alkane sulfonate, sodium alkyl diphenyl disulfonate, sodium alkyl naphthalene sulfonate, sodium arylsulfonate formaldehyde condensate, sodium beta Naphthalene sulfonate formaldehyde condensate, sodium dialkyl sulfosuccinate, sodium dodecylbenzene sulfonate, sodium fatty alcohol sulfate, sodium lauryl sulfate, sodium polyoxyethylene alkylether sulfate, sodium polyoxyethylene lauryl ether sulfate, sodium soap of blended fatty acid, sodium soap of partially hydrogenated tallow fatty acid, sodium stearate, sorbitan distearate, sorbitan monolaurate, sorbitan monooleate, sorbitan monopalmitate, sorbitan monostearate, sorbitan sesquioleate, sorbitan trioleate, sorbitan tristearate, stearyl amine acetate, stearyl trimethyl ammonium chloride, triethanolamine lauryl sulfate, and triethanolamine polyoxyethylene alkylether sulfate.

Another use for the Larrea leaf resin extract prepared using the method of the present invention includes mixing the Larrea extract with therapeutically effective amounts of at least one anti-inflammatory corticosteroid compound. This may be accomplished by a direct mixing of the corticosteroid compound, preferably prednisone for oral formulations and betamethasone dipropionate for topical formulations, at the proper concentration, generally 0.1 to 2.5% for topically applied corticosteroids and generally 5 to 60 milligrams per day for orally ingested steroids, with a liquid base, lotion base, a cream base, or a mixture of dry excipient ingredients containing the Larrea extract. To assist with uniform dispersion of the corticosteroid compound into the liquid base, lotion base, cream base or dry excipient ingredients, the corticosteroid compound may be dissolved or dispersed in a suitable, pharmaceutically acceptable solvent, preferably USP/NF grade anhydrous ethanol, prior to mixing in to the liquid base, lotion base, cream base or dry excipient ingredients. Additionally, the mixture resulting from the formulations containing dry excipient materials can be prepared in a manner suitable for milling into a uniform powder and encapsulation in standard, hard gelatin capsules.

Still a further aspect of the present invention includes addition of a local anesthetic to the Larrea extract at a therapeutically effective concentration. This may be accomplished by direct mixing of the anesthetic compound, preferably lidocaine base, at the proper concentration, generally 1 to 30% concentration, with a liquid base, lotion base or cream base containing the Larrea extract. To assist with uniform dispersion of the anesthetic compound, the anesthetic compound may be dissolved or dispersed in a suitable, pharmaceutically acceptable solvent, preferably USP/NF grade isopropanol, prior to mixing into the liquid base, lotion base, or cream base. Liquid, lotion and cream based formulations containing pharmaceutically ingredients may be used for topical application to regions of the body affected by herpes virus infections.

Another aspect of the present invention includes addition of a non-steroidal anti-inflammatory compound (NSAID) at a therapeutically effective concentration to the concentrated Larrea extract. This may be accomplished by direct mixing of the NSAID, preferably benzydamine at 3% (weight/weight) concentration for topical formulations, and ibuprofen at 100 milligrams per 50 milligrams of Larrea extract for oral formulations, with a liquid base, lotion base, cream base or dry excipient ingredients containing the Larrea extract. To assist with uniform dispersion of the NSAID into the liquid base, lotion base, cream base or dry excipient ingredients, the NSAID may be dissolved or dispersed in a suitable, pharmaceutically acceptable solvent, preferably USP/NF grade ethanol, prior to mixing in to the liquid base, lotion base, cream base or dry excipient ingredients. Additionally, the mixture resulting from the formulations containing dry excipient materials can be prepared in a manner suitable for milling into a uniform powder and encapsulation in standard gelatin capsules or compression into tablets.

Another aspect of the present invention includes addition of a lipid or lipid containing substance at a therapeutically effective concentration, to the concentrated Larrea extract. The lipid or lipid containing substance may include at least one of the anti-inflammatory fatty acids, GLA, EPA, DHA, and LNA. This can be accomplished by direct mixing of the lipid or lipid containing substance, preferably marine fish oils that are rich in EPA and DHA or flax seed oil which is rich in LNA, at about 1 to 20 grams per day for oral formulations, and about 1 to 80% (weight/weight) for topically applied formulations, with a liquid base, lotion base, cream base or dry excipient ingredients containing the Larrea extract. Additionally, the resulting liquid formulations may be encapsulated in soft gelatin capsules and the mixture resulting from the formulations containing dry excipient materials can be prepared in a manner suitable for milling into a uniform powder and subsequent tableting or encapsulation in standard gelatin capsules or compression into tablets.

Yet another aspect of the present invention includes addition of a substance P antagonist at a therapeutically effective concentration, to the concentrated Larrea extract. This can be accomplished by direct mixing of the substance P antagonist, preferably natural capsaicin, at the proper therapeutic concentration, generally 0.025 to 0.1%, with a liquid base, lotion base or cream base containing the Larrea extract. To assist with uniform dispersion of the substance P antagonist into the liquid base, lotion base or cream base, the substance P antagonist, preferably natural capsaicin, may be dissolved or dispersed in a suitable, pharmaceutically acceptable solvent, preferably USP/NF grade isopropanol, prior to mixing in to the liquid base, lotion base or cream base. Liquid, lotion and cream based formulations containing pharmaceutically ingredients may be used for topical application to regions of the body affected by herpes virus infections.

Further, experiments performed by the present inventor have shown that formulations based on the Larrea extract prepared according to the method of the present invention have pronounced antiviral activity against Herpes simplex virus types 1 and 2, and against Kaposi's Sarcoma in human patients. Experimentation has also shown that the described formulations have pronounced antiviral activity against Herpes simplex virus type 1 (HSV-1) in both animal cell culture models and human volunteers as well as anti-inflammatory action in human volunteers.

Also, as previously mentioned, the Larrea extract prepared according to the above described methods, including the phenylic compounds (predominantly lignans and flavonoids) contained therein, can function as an inhibitor of human aromatase enzymes. Based on the composition of lignans and flavonoids in Larrea extract and the fact that lipophyllic compounds such as lignans and flavonoids tend to concentrate in fatty tissues, such as adipose tissue, a pharmaceutically useful dosage for the partial inhibition of aromatase enzymes in human preadipocyte tissues can be as low as 100 to 500 milligrams of Larrea extract per day taken over the course of several years prior to the onset of menopause. In this way, supplementation with a small dose of Larrea extract, only several hundred milligrams per day prior to the onset of menopause, can significantly decrease aromatase levels in human preadipocyte tissues and thereby prevent or diminish the incidence of breast cancer in these individuals.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

1. A method of preparing a nontoxic composition that includes an extract of Larrea tridentata external leaf resin and is substantially free of nordihydroguaiaretic acid (NDGA) quinone, the method comprising the steps of:
mixing dried, whole Larrea tridentata leaves with an aqueous solvent to produce an extract; and
reducing any NDGA quinone in the extract to NDGA.

2. The method of claim 1, wherein the reducing step comprises saturating the extract with ascorbic acid.

3. The method of claim 1, wherein the aqueous solvent comprises a mixture of a polar organic solvent and water.

4. The method of claim 3, wherein the organic solvent is at least one solvent selected from the group consisting of propylene glycol, glycerin, and ethanol.

5. The method of claim 1, further comprising heating the aqueous solvent.

6. The method of claim 1, wherein approximately 1 liter of the aqueous solvent is mixed for every approximately 1 kg of the Larrea tridentata plant material.

7. The method of claim 1, wherein mixing is performed for approximately two to approximately six hours.

8. The method of claim 1, further comprising the step of:
separating the extract from the Larrea tridentata plant material.

9. The method of claim 8, wherein the separating step comprises decanting the extract from the Larrea tridentata plant material.

10. The method of claim 8, wherein the separating step comprises centrifuging the mixed extract and Larrea tridentata plant material.

11. The method of claim 8, further comprising the steps of:
rinsing the separated Larrea tridentata plant material with an aqueous solution comprising at least one component selected from the group consisting of a reducing agent, an alkaline pH adjusting agent, and a detergent; and
collecting the aqueous solution with the extract after rinsing the Larrea tridentata plant material.

12. The method of claim 11, wherein the aqueous solution comprises ascorbic acid.

13. The method of claim 11, wherein the aqueous solution comprises polysorbate 80.

14. The method of claim 11, wherein the aqueous solution comprises sodium hydroxide.

15. The method of claim 1, further comprising the step of:
standardizing the concentration of leaf resin in the extract, including measuring absorbance of the extract at 370 run.

16. The method of claim 1, further comprising the step of:
precipitating leaf resins from the extract.

17. The method of claim 16, wherein precipitating the leaf resins comprises adjusting the extract to an acidic pH.

18. The method of claim 16, wherein precipitating the leaf resins comprises adding a water soluble salt to the extract.

19. The method of claim 16, wherein precipitating the leaf resins comprises cooling the extract.

20. A method of preparing a nontoxic composition that includes an extract of Larrea tridentata plant material and is substantially free of nordihydroguaiaretic acid (NDGA) quinone, the method comprising the steps of:
mixing the Larrea tridentata plant material with an aqueous solvent to produce an extract;
reducing any NDGA quinone in the extract to NDGA;
separating the extract from the Larrea tridentata plant material;
rinsing the separated Larrea tridentata plant material with an aqueous solution comprising at least one component selected from the group consisting of a reducing agent, an alkaline pH adjusting agent, and a detergent; and
collecting the aqueous solution with the extract after rinsing the Larrea tridentata plant material.

21. The method of claim 20, further comprising the step of:
precipitating leaf resins from the extract.

22. The method of claim 21, wherein precipitating the leaf resins comprises adjusting the extract to an acidic pH, and adding a water soluble salt to the extract.

23. The method of claim 22, wherein the aqueous solution used in the rinsing step comprises ascorbic acid.

24. The method of claim 20, wherein the aqueous solvent comprises a mixture of a polar organic solvent and water.

25. A method of treating an infection from a virus that has replication affected by transcription nuclear factor-kappa beta (NF-kB) activation, the method comprising administering to a human in need thereof a pharmaceutical composition comprising an extract of Larrea tridentata in an amount that is effective to suppress NF-kB activation.

26. The method of claim 25, wherein the virus being treated is from the family Paramyxoviridae.

27. The method of claim 26, wherein the virus being treated is respiratory syncytial virus.

28. The method of claim 25, wherein the virus being treated is from the family Orthomyxoviridae.

29. The method of claim 28, wherein the virus being treated is an influenza virus.

30. The method of claim 25, wherein the virus being treated is from the family Adenoviridae.

31. The method of claim 30, wherein the virus being treated is an adenovirus.

32. The method of claim 25, wherein the virus being treated is from the family Picorniviridae.

33. The method of claim 32, wherein the virus being treated is selected from the group consisting of rhinoviruses and hepatitis A viruses.

34. The method of claim 25, wherein the virus being treated is from the family Hepadnaviridae.

35. The method of claim 34, wherein the virus being treated is a hepatitis B virus.

36. The method of claim 25, wherein the virus being treated is from the family Flaviviridae.

37. The method of claim 36, wherein the virus being treated is a hepatitis C virus.

38. An antimicrobial solution, comprising:
an aqueous solvent; and
an extract of Larrea tridentata plant material.

39. The antimicrobial solution of claim 38, further comprising a reducing agent.

40. The antimicrobial solution of claim 38, further comprising at least one component selected from the group consisting of isopropanol, ethanol, propanol, butanol, propylene glycol, ethylene glycol, polyethylene glycol, polypropylene glycol, butylene glycol, 2-alkyl-N-carboxymethyl-N-hydroxyethyl imidazolinium betaine, alkyl alkanol amide, alkyl benzyl dimethyl ammonium chloride, ammonium lauryl sulfate, cetyl trimethyl ammonium chloride, coconut amine acetate, distearyl dimethyl ammonium chloride, ethylene glycol distearate, fatty acid alkanol amide, glycerol monooleate, lauryl betaine, lauryl dimethyl amine oxide, lauryl imidazolinium betaine, lauryl trimethyl ammonium chloride, mono- and diglyceride, polyethylene glycol distearate, polyethylene glycol monooleate, polyethylene glycol monolaurate, polyethylene glycol monostearate, polyoxyalkylene alkylether, polyoxyethylene alkyl amine, polyoxyethylene alkyl ether, polyoxyethylene cetyl ether, polyoxyethylene derivatives, polyoxyethylene hydrogenated castol oil, polyoxyethylene lauryl ether, polyoxyethylene oleyl ether, polyoxyethylene sorbitan mono coconut fatty acid ester, polyoxyethylene sorbitan monolaurate, polyoxyethylene sorbitan monooleate, polyoxyethylene sorbitan monopalmitate, polyoxyethylene sorbitan monostearate, polyoxyethylene sorbitan triisostearate, polyoxyethylene sorbitan trioleate, polyoxyethylene sorbitan tristearate, polyoxyethylene sorbitol tetraoleate, polyoxyethylene stearyl ether, potassium hydroxy stearate, potassium oleate, potassium polyoxyethylene alkyl ether phosphate, potassium salt of castor oil, potassium soap of partially hydrogenated tallow fatty acid, sodium alkane sulfonate, sodium alkyl diphenyl disulfonate, sodium alkyl naphthalene sulfonate, sodium arylsulfonate formaldehyde condensate, sodium beta Naphthalene sulfonate formaldehyde condensate, sodium dialkyl sulfosuccinate, sodium dodecylbenzene sulfonate, sodium fatty alcohol sulfate, sodium lauryl sulfate, sodium polyoxyethylene alkylether sulfate, sodium polyoxyethylene lauryl ether sulfate, sodium soap of blended fatty acid, sodium soap of partially hydrogenated tallow fatty acid, sodium stearate, sorbitan distearate, sorbitan monolaurate, sorbitan monooleate, sorbitan monopalmitate, sorbitan monostearate, sorbitan sesquioleate, sorbitan trioleate, sorbitan tristearate, stearyl amine acetate, stearyl trimethyl ammonium chloride, triethanolamine lauryl sulfate, and triethanolamine polyoxyethylene alkylether sulfate.

41. A formulation for medical and health products, comprising:
an extract of Larrea tridentata plant material; and
at least one detoxification enhancer that functions as a chemoprotective agent.

42. The formulation of claim 41, wherein the at least one detoxification enhancer that functions as a chemoprotective agent is selected from the group consisting of diindolymethane, indole-3-carbinol, methionine, methionine derivatives, cysteine, cysteine derivatives, N-acetyl cysteine, cystine, methylsulfonylmethane (MSM), flavonoids, milk thistle extract, silymarin, sylbinin, benzothiazole, garlic extract, propolis, tannic acid, ellagic acid, coumarin, turmeric extract, curcumin, allyl isothiocyanate, benzyl isothiocyanate, phenethyl isothiocyanate, benzyl thiocyanate, brassica extracts, cruciferous vegetable extracts, broccoli extract, brussel sprout extract, sulforaphane, sulforaphane nitrile, glucosinolates, green tea extract, epigallocatechin gallate (EGCG), tannic acid, ginkgo extract, resveratrol, grape extract, selenium, selenomethionine, glutathione, lipoic acid, coenzyme Q, oleuropein, hops extract, humulone and/or synthetic phenylic antioxidants such as BHA, BHT, TBHQ and hydroquinone.

43. The formulation of claim 42, further comprising at least one stimulant selected from the group consisting of coffee extract, gotu kola, cola extract, caffeine, cocoa extract, theobromine, tea extract, theophylline, synephrine, ginseng, taurine, ephedrine, and pseudoephedrine.

44. A formulation for preservation of construction materials containing cellulose against damage by insects, fungi or other pests, comprising:
an extract of Larrea tridentata plant material; and
at least one aqueous solvent capable of facilitating penetration of the extract into the construction material.

45. The formulation of claim 44, further comprising at least one additional ingredient from the group consisting of urethane, acrylic, shellac, latex, epoxy, enamel, varnish, coal tar pitch and coal tar pitch emulsion, pigments, clay, and plastic polymers.

46. A solid construction material resistant to damage by insects, fungi or other pests, the construction material comprising:
cellulose as a structural base; and
an extract of Larrea tridentata plant material.

47. The solid construction material of claim 46, wherein the material is selected from the group consisting of paper and wood.

48. A formulation for preventing infestation of animals, insects, fungi, and bacteria, comprising:
an extract of Larrea tridentata plant material; and
at least one aqueous solvent.

49. The formulation of claim 46, further comprising at least one additional ingredient selected from the group consisting of isopropanol, ethanol, propanol, butanol, propylene glycol, ethylene glycol, polyethylene glycol, polypropylene glycol, butylene glycol, wood rosin, 2-alkyl-N-carboxymethyl-N-hydroxyethyl imidazolinium betaine, alkyl alkanol amide, alkyl benzyl dimethyl ammonium chloride, ammonium lauryl sulfate, cetyl trimethyl ammonium chloride, coconut amine acetate, distearyl dimethyl ammonium chloride, ethylene glycol distearate, fatty acid alkanol amide, glycerol monooleate, lauryl betaine, lauryl dimethyl amine oxide, lauryl imidazolinium betaine, lauryl trimethyl ammonium chloride, mono- and diglyceride, polyethylene glycol distearate, polyethylene glycol monooleate, polyethylene glycol monolaurate, polyethylene glycol monostearate, polyoxyalkylene alkylether, polyoxyethylene alkyl amine, polyoxyethylene alkyl ether, polyoxyethylene cetyl ether, polyoxyethylene derivatives, polyoxyethylene hydrogenated castol oil, polyoxyethylene lauryl ether, polyoxyethylene oleyl ether, polyoxyethylene sorbitan mono coconut fatty acid ester, polyoxyethylene sorbitan monolaurate, polyoxyethylene sorbitan monooleate, polyoxyethylene sorbitan monopalmitate, polyoxyethylene sorbitan monostearate, polyoxyethylene sorbitan triisostearate, polyoxyethylene sorbitan trioleate, polyoxyethylene sorbitan tristearate, polyoxyethylene sorbitol tetraoleate, polyoxyethylene stearyl ether, potassium hydroxy stearate, potassium oleate, potassium polyoxyethylene alkyl ether phosphate, potassium salt of castor oil, potassium soap of partially hydrogenated tallow fatty acid, sodium alkane sulfonate, sodium alkyl diphenyl disulfonate, sodium alkyl naphthalene sulfonate, sodium arylsulfonate formaldehyde condensate, sodium beta Naphthalene sulfonate formaldehyde condensate, sodium dialkyl sulfosuccinate, sodium dodecylbenzene sulfonate, sodium fatty alcohol sulfate, sodium lauryl sulfate, sodium polyoxyethylene alkylether sulfate, sodium polyoxyethylene lauryl ether sulfate, sodium soap of blended fatty acid, sodium soap of partially hydrogenated tallow fatty acid, sodium stearate, sorbitan distearate, sorbitan monolaurate, sorbitan monooleate, sorbitan monopalmitate, sorbitan monostearate, sorbitan sesquioleate, sorbitan trioleate, sorbitan tristearate, stearyl amine acetate, stearyl trimethyl ammonium chloride, triethanolamine lauryl sulfate, and triethanolamine polyoxyethylene alkylether sulfate.
